(19) United States
(12) Patent Application Publication
(10) Pub. No.: US 2012/0231087 A1
(43) Pub. Date: Sep. 13, 2012

(54) Compositions And Methods for Nutritional Supplementation

(75) Inventors: Inge Bruheim, Volda (NO); Mikko Griinari, Espoo (FI); Stig Rune Remoy, Fosnavag (NO)

(73) Assignee: OLYMPIC SEAFOOD

(21) Appl. No.: 13/413,523

(22) Filed: Mar. 6, 2012

Related U.S. Application Data
(60) Provisional application No. 61/450,066, filed on Mar. 7, 2011.

(51) Int. Cl. A61K 38/17; A61K 33/32; A61K 33/30; A61K 33/34; A61K 36/258; A61K 36/82; A61K 36/906; A61K 36/8962; A61K 36/87; A61K 38/16; A61K 36/064; A61K 36/38; A61K 36/886; A61K 36/16; A61P 19/02; A61K 35/56

(52) U.S. Cl. 424/547; 514/16.8; 424/639; 424/643; 424/638; 424/728; 424/729; 424/756; 424/754; 424/766; 514/5.6; 424/195.16; 424/730; 424/744; 424/752

(57) ABSTRACT

The present invention relates to nutritional supplement compositions comprising krill meal and lipid insoluble ingredients. The compositions can be used to provide supplementary nutrition to humans or animals. The present invention relates also formulations, such as tablets, which are easy to administer to animals. It is also shown that krill meal added to animal food at low levels will improve the palatability of the product.

### FIELD OF THE INVENTION

The present invention relates to compositions useful as nutritional supplements and methods of production. The invention relates also to methods of adding krill meal to food or feed for improving palatability. For example, krill oil in the form of krill meal can be mixed with nutrients, in particular lipid insoluble nutrients. The compositions prepared by mixing krill meal and lipid insoluble nutrients are homogenous and stable.

### BACKGROUND OF THE INVENTION

Companion animals (e.g., domestic pets) are typically fed complete diets (dry or wet) formulated to provide sufficient amount of nutrients to satisfy the animal's needs for maintenance and activity. Young animals may have special nutritional needs for growth, which can be satisfied by specially formulated diets. Besides the basic nutritional needs, companion animals may have special needs that can be satisfied with specific nutrients supplied by nutritional supplements.

In order to overcome the problem of administering the supplements containing combinations of nutrients it has been disclosed previously [5] a method of including the nutrients in chews made of rawhide. The problem with this approach to combine different nutrients is that the nutritional product is bulky and requires the animal to want to chew the product in order to obtain the nutrients. It is true that many dogs like to chew, but this is not a universal feature among dogs. Many dogs have a poor appetite and they may have poor teeth that both hinder the delivery of important nutrients as a daily routine. Capsulated oil products for companion animals are accompanied with instructions that recommend to break the capsule and to empty the contents of the capsule into a bowl of food.

Thus, there is a need for products by which supplementary nutrients, in particular nutrients comprising oil and lipid insoluble nutrients, could be administered to objects in need for said nutrients. Such nutrients would be useful in particular to promote the health of companion animals, but also other animals, such as domestic animals, or humans.

### SUMMARY OF THE INVENTION

The present invention relates to compositions useful as nutritional supplements and methods of production. The invention relates also to methods of adding krill meal to food or feed for improving palatability. For example, krill oil in the form of krill meal can be mixed with nutrients, in particular lipid insoluble nutrients. The compositions prepared by mixing krill meal and lipid insoluble nutrients are homogenous and stable.

In one embodiment, the present invention contemplates a nutritional supplement composition comprising krill meal and lipid insoluble ingredients. In one embodiment, the composition is homogenous. In one embodiment, the krill meal comprises a krill oil. In one embodiment, the composition further comprises at least one omega-3 fatty acid. In one embodiment, the composition is stable. In one embodiment, the homogeneity of the composition is characterized by a lack of phase separation. In one embodiment, the stability of the composition is characterized by, in comparison to krill oil, lower lyso-phospholipids, increased omega-3 levels, lower peroxide, lower p-anisidine, and higher astaxanthin. In one embodiment, the lipid insoluble ingredient comprises cartilage protecting substances. In one embodiment, the cartilage protecting substances are glucosamine and chondroitin. In one embodiment, the lipid insoluble ingredients are selected from the group consisting of glucosamine hydrochloride, glucosamine sulfate, glucosamine potassium, glucosamine sodium, N-acetyl d-glucosamine, chondroitin, chondroitin sulfate, curcumin, ascorbic acid, hyaluronic acid, green lipped mussel powder, creatine, L-carnitine, ascorbic acid, manganese, manganese proteinate, zinc, zinc proteinate, copper, copper proteinate, ginseng, green tea extract, ginger, garlic, vincamine, grape seed extract, grape seed meal, dimethyl glycine, whey protein, brewer's yeast, St. John's wort, vinpocetine, aloe vera, ginko biloba, curcumin, betaglucans, and mannaoligosaccharides. In one embodiment, the composition comprises a krill meal to lipid insoluble ingredients weight ratio ranging between approximately 16:1 to 1:1. In one embodiment, the composition further comprises a lipid soluble ingredient. In one embodiment, the lipid soluble ingredient is selected from the group consisting of vitamin A, vitamin D, vitamin E, alpha lipoic acid, lutein, and natural carotenoids. In one embodiment, the composition comprises 10-90% (w/w) krill meal, 10-90% (w/w) lipid insoluble ingredient(s), and 0-80% (w/w) excipients. In one embodiment, the composition comprises a suitable form selected from the group consisting of a tablet, a capsule, a granule, a pellet, a powder, or a pet treat. In one embodiment, the capsule comprises a hard gelatin capsule. In one embodiment, the hard gelatin capsule is a sprinkle capsule. In one embodiment, the pet treat comprises at least one flavor ingredient. In one embodiment, the pet treat comprises a filler ingredient. In one embodiment, the composition further comprises an excipient. In one embodiment, the excipient is selected from the group consisting of diluents, fillers, binders, granulating agents, adhesives, disintegrants, lubricants, antiadherants, glidants, wetting agents, dissolution retardants, enhancers, adsorbents, buffers, chelating agents, preservatives, colours, flavours, sweeteners, starch, pregelatinized starch, maltodextrin, monohydrous dextrose, alginic acid, sorbitol, mannitol, magnesium stearate, stearic acid, talc, silic, cellulose, microcrystalline cellulose, methyl cellulose, polyvinylpyrrolidone and—commercial products, such as Aerosil®, Kollidon® and Explotab®.

In one embodiment, the present invention contemplates a composition comprising an animal food and a krill meal food ingredient. In one embodiment, the krill meal food ingredient comprises a krill oil. In one embodiment, the composition further comprises at least one omega-3 fatty acid. In one embodiment, the krill meal food ingredient is less than 5% of said composition. In one embodiment, the krill meal food ingredient is less than 1% of said composition.

In one embodiment, the present invention contemplates a method, comprising: a) providing: i) an animal exhibiting at least one symptom of a degenerative joint disease; ii) a nutritional supplement composition comprising krill meal and a lipid insoluble ingredient; b) administering said composition to said animal under conditions such that said at least one symptom is reduced. In one embodiment, the composition is homogenous. In one embodiment, the krill meal comprises a krill oil. In one embodiment, the composition further comprises at least one omega-3 fatty acid. In one embodiment, the lipid insoluble ingredient is stable. In one embodiment, the homogeneity of the composition is characterized by a lack of phase separation. In one embodiment, the stability of the composition is characterized by, in comparison to krill oil, lower lyso-phospholipids, increased omega-3 levels, lower peroxide, lower p-anisidine, and higher astaxanthin. In one embodiment, the at least one symptom comprises pain. In one embodiment, the at least one symptom comprises stiffness. In one embodiment, the at least one symptom comprises lameness. In one embodiment, the at least one symptom comprises mobility. In one embodiment, the at least one symptom comprises mood. In one embodiment, the at least one symptom comprises activity. In one embodiment, the at least one embodiment comprises play. In one embodiment, mobility is assessed by a modified Helsinki Chronic Pain Index. In one embodiment, the lameness is determined by a deviation of a 60:40 weight distribution between the front legs and the rear legs of a dog. In one embodiment, the administered composition comprises krill meal ranging between 0.005-0.50 grams per day per kilogram of said animal's body weight. In one embodiment, the duration of the administration comprised at least six weeks. In one embodiment, the degenerative joint disease comprises osteoarthritis. In one embodiment, the lipid soluble ingredient comprises cartilage protecting substances. In one embodiment, the cartilage protecting substances are glucosamine and chondroitin. In one embodiment, the lipid insoluble ingredients are selected from the group consisting of glucosamine hydrochloride, glucosamine sulfate, glucosamine potassium, glucosamine sodium, N-acetyl d-glucosamine, chondroitin, chondroitin sulfate, curcumin, ascorbic acid, hyaluronic acid, green lipped mussel powder, creatine, L-carnitine, ascorbic acid, manganese, manganese proteinate, zinc, zinc proteinate, copper, copper proteinate, ginseng, green tea extract, ginger, garlic, vincamine, grape seed extract, grape seed meal, dimethyl glycine, whey protein, brewer's yeast, St. John's wort, vinpocetine, aloe vera, ginko biloba, curcumin, betaglucans, and mannaoligosaccharides. In one embodiment, the composition comprises a krill meal to lipid insoluble ingredients weight ratio ranging between approximately 16:1 to 1:1. In one embodiment, the composition further comprises a lipid soluble ingredient. In one embodiment, the lipid soluble ingredient is selected from the group consisting of vitamin A, vitamin D, vitamin E, alpha lipoic acid, lutein, and natural carotenoids. In one embodiment, the composition comprises 10-90% (w/w) krill meal, 10-90% (w/w) lipid insoluble ingredient(s), and 0-80% (w/w) excipients. In one embodiment, the composition comprises a suitable form selected from the group consisting of a tablet, a capsule, a granule, a pellet, a powder, or a pet treat. In one embodiment, the capsule comprises a hard gelatin capsule. In one embodiment, the hard gelatin capsule is a sprinkle capsule. In one embodiment, the pet treat comprises at least one flavor ingredient. In one embodiment, the pet treat comprises a filler ingredient. In one embodiment, the composition further comprises an excipient. In one embodiment, the excipient is selected from the group consisting of diluents, fillers, binders, granulating agents, adhesives, disintegrants, lubricants, antiadherants, glidants, wetting agents, dissolution retardants, enhancers, adsorbents, buffers, chelating agents, preservatives, colours, flavours, sweeteners, starch, pregelatinized starch, maltodextrin, monohydrous dextrose, alginic acid, sorbitol, mannitol, magnesium stearate, stearic acid, talc, silic, cellulose, microcrystalline cellulose, methyl cellulose, polyvinylpyrrolidone and—commercial products, such as Aerosil®, Kollidon® and Explotab®.

In one embodiment, the present invention contemplates a method, comprising: a) providing: i) an animal in need of increased food intake; ii) an animal food or feed comprising between approximately 0.01% (w/w) and 1.0% (w/w) krill meal; b) feeding said animal said food under conditions such that said animal's food intake increases. In one embodiment, the krill meal comprises krill oil. In one embodiment, the krill oil comprises at least one omega-3 fatty acid. In one embodiment, the method further comprises improving the health of said animal. In one embodiment, the animal comprises a non-human animal. In one embodiment, the non-human animal comprises a dog.

### Definitions

As used herein and in the appended claims, the singular forms “a,” “an,” and “the” include plural reference unless the context clearly dictates otherwise, e.g., reference to “a method” includes a plurality of such methods.

The words “comprise”, “comprises”, and “comprising” are to be interpreted inclusively rather than exclusively.

The term “optionally” means here the same as “possibly”. For example, compositions disclosed herein as “optionally comprises excipients”, means that the composition may or may not comprise excipients, in other words the composition possibly comprises excipients.

The term “animal” as used herein, means species including but not limited to mammals, fish, crustaceans, amphibians, reptiles etc. In particular, a “companion animal” refers to any non-human animal kept by a human as a pet or any animal of a variety of species that have been widely domesticated as pets, such as dogs (Canis familiaris), and cats (Felis domesticus), whether or not the animal is kept solely or partly for companionship. Companion animals also include working animals including but not limited to horses, cows, pigs, goats, sheep, dogs (i.e., for example, livestock herding) and/or cats (i.e., for example, rodent control).

The term “krill meal” as used herein, refers here to any mixture of proteins and lipids derived from krill. The term is not limited to any particular method of making krill meal, but any method known in the art is contemplated.

The term “effective amount” refers to any amount of a supplement that improves the palatability of the food or feed.

The term “ingredient” or “supplement” refers to any composition can be formulated to a suitable form, such as a tablet, a granule, a pellet or powder. The composition may be formulated also to a pet treat or a hard gelatin capsule (sprinkle capsule) can be filled with the composition.

As used herein, the term “omega-3 fatty acid” refers to fatty acids which have the final double bond between the third and the fourth carbon atom counting from the methyl end of the carbon chain. Omega-3 fatty acids mainly concerned in this disclosure are the long chain polyunsaturated fatty acids eicosapentaenoic acid (EPA) and docospentaenoic acid (DHA) as well as the minor omega-3 fatty acids including eicosatetraenoic acid (ETA) and docosapentaenoic acid (DPA).

As used herein, the term “lipid insoluble ingredient” refers to any compound not soluble in a lipophilic solvent such as chloroform, n-hexane and toluene. Preferably such compounds are St. John's wort, creatine, dimethyl glycine, ginko biloba, ginseng, betaglucan, mannaoligosaccharides, vincamine, whey protein, vinpocetine, zinc, zinc proteinate, copper, copper proteinate.. More preferably such compounds are grape seed extract, grape seed meal, ginger, garlic, aloe vera and green lipped mussel powder and brewer's yeast. Even more preferably such compounds are L-carnitine, hyaluronic acid and green tea extract. Most preferably such compounds are glucosamine hydrochloride, glucosamine sulfate, glucosamine potassium, glucosamine sodium, N-acetyl d-glucosamine, chondroitin sulfate, curcumin, ascorbic acid, manganese and manganese proteinate.

As used herein, the term “glucosamine” refers to 2-Amino-2-deoxy-D-glucose chitosamine or any derivative thereof.

As used herein, the term “chondroitin” refers to chondroitin sulfate, which is a sulfated glycosaminoglycan composed of a chain of alternating sugars (N-acetylgalactosamine and glucuronic acid) or any derivative thereof including low molecular weight forms.

Lipid insoluble ingredients may also comprise any combination of the mentioned substances. Preferred lipid insoluble ingredients comprise cartilage protecting substances such as glucosamine and/or chondroitin.

The term “excipients”, as used herein, refer to any substance needed to formulate the composition to the desired form. For example, suitable excipients include but are not limited to, diluents or fillers, binders or granulating agents or adhesives, disintegrants, lubricants, antiadherants, glidants, wetting agents, dissolution retardants or enhancers, adsorbents, buffers, chelating agents, preservatives, colours, flavours and sweeteners. Typical excipients are for example starch, pregelatinized starch, maltodextrin, monohydrous dextrose, alginic acid, sorbitol and mannitol. In general, the excipient should be selected from non-toxic excipients (IIG, Inactive Ingredient Guide, or GRAS, Generally Regarded as safe, Handbook of Pharmaceutical Excipients). Typical excipients in particular for tableting are for example magnesium stearate, stearic acid, talc, silic, cellulose, microcrystalline cellulose, methyl cellulose, polyvinylpyrrolidone and—commercial products, such as Aerosil®, Kollidon® and Explotab®. Excipients can be added into the direct powder compression formula.

As used herein, the following terms are referred to in relation to krill meal use in various formulations:
“animal feed” refers to any mixture of animal feed ingredients providing energy and nutrient requirements (e.g., protein, fat, carbohydrates, minerals and micronutrients) in a balanced ratio. For example, a daily intake of ‘animal feed’ for companion animals is typically between 1-2% of body weight, for example 300 g/d for a 20 kg dog;
“animal feed ingredient” refers to any organic or mineral component added to an animal feed mixture in appreciable amounts (i.e., for example a bulk ingredient). Animal feed ingredients are usually added to animal feed at levels greater than 1% (w/w), preferably 1 to 90% (w/w), typically 10 to 50% (w/w);
“flavor ingredient” is typically an organic component added to an animal feed for the purposes of improving feed palatability. Flavor ingredients are usually added to animal feeds at levels preferably less than 1% (w/w), more preferably less than 0.5%, most preferably less than 0.1% by weight of the animal feed;
“nutritional supplement” refers to any mixture of specific ingredients that may be of short supply in routinely provided food. For example, a daily intake of a nutritional supplement is typically <1 gram to few grams; the form of a nutritional supplement for companion animals can be liquid or dry (e.g., pellet, tablet, granule, powder, capsule etc.);
“treat” refers to any nutritional product form given to a companion animal as a reward or a snack between meals. Treats may contain food ingredients and other ingredients that give a special consistency to the treat e.g. chewable treat. Treats are not usually considered important for their nutritional content. Treats may be used as rewards and should be highly palatable. Alternatively, treats may also be used as sources of supplementary nutrition and should have ingredients that improve the overall nutritional balance. For example, ‘healthy treats’ are a special form of treats that contain specific health promoting ingredients; daily intake of a treat is typically from several grams per day up to 100 g/d for an average sized dog (20 kg body weight).

### DETAILED DESCRIPTION OF THE INVENTION

The present invention relates to compositions useful as nutritional supplements and methods of production. The invention relates also to methods of adding krill meal to food or feed for improving palatability. For example, krill oil in the form of krill meal can be mixed with nutrients, in particular lipid insoluble nutrients. The compositions prepared by mixing hill meal and lipid insoluble nutrients are homogenous and stable.

One embodiment of the invention provides a composition comprising krill meal and lipid insoluble ingredients for use as a nutritional supplement. The composition may further comprise excipients suitable for consumption by an animal (i.e., for example, a companion animal or a human). The composition is particularly suitable for companion animals, such as dogs or cats.

One embodiment of the present invention provides a method for improving the palatability of food or feed. The data presented herein demonstrates that krill meal in nutraceutical formulations is highly palatable for animals (e.g., dogs) at very low doses. In one embodiment, improving the palatability of food or feed with krill meal increases an animal's intake of food thereby providing a mechanism for improved health.

One embodiment of the present invention provides a method for producing a krill meal nutritional supplement composition.

### I. Krill Meal

In one embodiment, the present invention contemplates a krill meal. In one embodiment, the krill meal comprises krill oil, wherein the krill oil can be mixed/combined with lipid insoluble nutrients. In one embodiment, the krill meal is formulated into a mixture of nutrients, krill oil and standard excipients. In one embodiment, the mixture comprises a composition formulated as a tablet or a capsule. One advantage of krill meal compositions comprising krill oil, is that that the problem of extracting the krill oil is avoided. Extracting krill oil is generally a difficult process resulting in high cost of the oil and often inferior quality (e.g., lower levels of long chain PUFA omega-3) compared to the oil in the meal. [7,8].

Antarctic krill (Euphausia superba) is a small shrimp like crustacean which can be found in huge quantities in the Southern Ocean, off the coast of Antarctica. In order to adapt to an ever changing supply of food, krill has developed a unique survival strategy comprising highly potent digestive enzymes to facilitate complete and rapid breakdown ingested food. However, these potent digestive enzymes also result in a rapid breakdown of proteins and lipids (proteolysis and lipolysis, respectively) after harvesting of the krill. For this reason the nutrient content of krill varies, depending on harvesting area and season. In order to minimize post-harvest enzymatic activity, the krill can be frozen or turned into a meal immediately after harvesting (i.e., for example, on board the fishing vessel).

The lipids found in krill are rich in phospholipids and especially phosphatidyl choline with high content of long chain omega-3 fatty acids, in addition to the antioxidant astaxanthin. Gentle processing methods enable the levels of these nutrients to remain stable throughout the process.

Previously, a number of processes for making krill meal have been disclosed. For example krill meal can be obtained by cooking krill, followed by pressing, drying and milling, using the same technology and principles as for making fish meal. Alternatively, the krill can be cooked at two different temperatures, where the majority of the phospholipids are removed at a lower temperature [12]. Other methods of making krill meal have been disclosed, where a dried powdery and granular krill product containing all components of krill was obtained. [13]. The production process comprised the steps of lightly dehydrating krill, coarsely crushing the krill, and drying the coarsely crushed krill under heating. Other known methods include the use of freeze drying followed by milling [14] and the use of alcohol to denature the enzymes [15]. Alternatively, a protein rich krill meal can be obtained by enzymatic hydrolysis as disclosed in [16-17].

### II. Krill Meal Nutritional Supplement Compositions

Long chain polyunsaturated omega-3 fatty acids (omega-3 FA) present in fish oil are considered beneficial supplementary nutrients for companion animals e.g. dogs. Besides fish, omega-3 fatty acids can be derived from several sources of marine organisms including micro algae, seal, squid, molluscs, krill to name a few. Typical sources of omega-3 fatty acids from marine organisms is the oil extracted from the organism.

It is also known in the art to use meal from a marine organism containing oil as the source of omega-3 fatty acids in the animal diets [1]. Supplementary nutrients are often used in combination to provide synergistic effects. For example the cartilage protecting nutrients, such as glucosamine and chondroitin, are often used in combination with omega-3 fatty acids to provide nutritional support for joints, both healthy joints and those inflicted by degenerative joint disease [2-4]. Lipids extracted from Antarctic krill (Euphausia superba) or hill oil contains omega-3 fatty acids.

Krill oil has been used as an ingredient in nutritional supplements with remarkable potential to alleviate the arthritis symptoms in human subjects [5]. Products involving combinations of krill oil and cartilage protecting supplements, such as Genflex 3 and Omegagen are commercially available for humans. It would be useful to have similar combination products for supplementing the diets of animals, such as dogs or other companion animals suffering from osteoarthritis. The form in which the combination products are presented i.e. in gelatin capsules is not the preferred form for supplementing health promoting nutrients to dogs, as dogs do not willingly eat the capsules.

Compositions comprising krill lipids in the form of krill meal and non-lipid soluble ingredients overcome a number of technological problems relating to combinations of nutrients that provide a basis for in vivo synergistic effects as a result of the nutrient combination. In some embodiments, a homogenous and stable composition is contemplated which does not separate into different phases over time. In some embodiments, the composition is formulated into a product comprising a tablet, a granule, a pellet or a powder.

It is well known in the prior art that capsulated material is difficult to administer to an uncooperative animal, in particular if the capsule is an unpalatable gelatin capsule. This invention provides a solution to this problem as it is shown that the novel compositions described herein are more willingly consumed by animals.

Simply combining krill oil and lipid insoluble nutrients such as glucosamine and chondroitin in a capsule, will result in sequestration of the lipid insoluble nutrients within the capsule. Further, the removal of sequestered nutrients from the capsule by squeezing the capsule is not possible without significant effort. The result is a situation where the daily administration of capsulated nutrients to the animal are not delivered efficiently. It is possible to combine oil and lipid insoluble nutrients also in a tablet, however, there is a limit for inclusion of oil in a tablet without extraordinary effort. [2, 6].

In one embodiment, the present invention contemplates a composition comprising one or more krill meal lipid insoluble ingredients, either alone or in combination. In one embodiment, the composition further comprises lipid soluble ingredients, for example vitamin A, vitamin D, vitamin E, alpha lipoic acid, lutein and other natural carotenoids. In one embodiment, the composition is formulated to form a tablet, a capsule, a granule, a pellet or powder. In one embodiment, the capsule is filled with the composition. In one embodiment, the capsule is a hard gelatin capsule. In one embodiment, the hard gelatin capsule is a sprinkle capsule. Although it is not necessary to understand the mechanism of an invention, it is believed that one advantage of a sprinkle capsule is that it is easily opened. In one embodiment, a sprinkle capsule comprising krill meal and a lipid insoluble ingredient in a powder form. Hard gelatin capsules are especially suitable as they can be opened easily by the companion animal owner and the contents dispensed to the animal, for example by sprinkling the composition on dog food.

In one embodiment, the krill meal composition comprises a pet treat. In one embodiment, the pet treat composition includes flavor ingredients. Although it is not necessary to understand the mechanism of an invention, it is believed that such flavor ingredients give the pet treat composition a special appealing flavour. In one embodiment, the flavor ingredients are either natural or synthetic. In one embodiment, the pet treat compositions further comprise filler ingredients. Although it is not necessary to understand the mechanism of an invention, it is believed that such filler ingredients provide a desirable consistency and bulk to the composition. In one embodiment, filler ingredients include but are not limited to, meat meals and extracts, other animal byproducts, fish meal and extracts, plant protein meals, cereal meals or fractions of cereals and various binding agents known in the art.

In one embodiment, a krill meal nutritional supplement composition comprises krill meal, lipid insoluble ingredients, omega-3 fatty acids, and excipients. In one embodiment, the krill meal ranges between approximately 10-90% (w/w) of the composition, more preferably ranges between approximately 20-60% (w/w) of the composition, and most preferably ranges between approximately 25 to 50% (w/w) of the composition. In one embodiment, the lipid insoluble ingredients range between approximately 10-90% (w/w) of the composition, more preferably ranges between approximately 20-60% (w/w) of the composition, and most preferably ranges between approximately 25 to 50% (w/w) of the composition. In one embodiment, the omega-3 fatty acids range between approximately at least 1% (w/w) to at least 5% (w/w) of the composition, and preferably at least 4% (w/w) of the composition. In one embodiment, the excipients range between approximately 0-80% (w/w) of the composition, more preferably ranging between approximately 0-60% (w/w) of the composition, and most preferably ranging between approximately 0 to 50% (w/w) of the composition, wherein the composition comprises about 20% (w/w) excipients

In one embodiment, a daily dose of a krill meal composition as disclosed herein is administered as between approximately 0.005 to 0.50 gram (g) krill meal/kilogram (kg) animal body weight, more preferably 0.01 to 0.25 g krill meal/kg animal body weight, and still more preferably 0.02 to 0.125 g krill meal/kg animal body weight. For example, when administering a krill meal composition to a dog of 20 kg body weight, a daily dose of the composition preferably would be between approximately 0.1-10 g krill meal per day per animal, more preferably between approximately 0.2 to 5 g/d/animal, still more preferably between approximately 0.4 to 2.5 g/d/animal.

In one embodiment, a daily dose of a krill meal composition as disclosed herein is between approximately 0.1-100 g per day per human individual, more preferably between approximately 0.25 to 50 g/d per human individual, and still more preferably between approximately 0.5 to 25 g/d per human individual.

In one embodiment, a tablet, granule, powder, pellet or capsule comprising a krill meal composition may be administered in a daily dose of between approximately 0.2 to 20 g/20 kg body weight, more preferably 0.4 to 10 g/20 kg body weight, still more preferably 0.8 to 5.0 g/20 kg body weight.

In one embodiment, the present invention contemplates a composition comprising krill meal and lipid insoluble ingredients in a weight ratio ranging between approximately 16:1 to 1:1. In one embodiment, the krill meal and lipid insoluble ingredient weight ratio is approximately 4:1.

In one embodiment, the present invention contemplates a composition comprising krill meal and krill oil in a weight ratio ranging between approximately 4:1 to 1:4. In one embodiment, the krill meal and krill oil weight ratio is approximately 1:1.

As described herein, animals (e.g., companion animals) prefer to consume food which contains added krill meal over food that does not contain krill meal. Companion animals have been shown to demonstrate preference to food that contains very low levels of krill meal as compared to food that does not contain krill meal. Although it is not necessary to understand the mechanism of an invention, it is believed that krill meal enhances the palatability of animal food or feed, in particular a companion animal's food or feed (i.e., for example, dog food) at concentrations that are at least an order of magnitude lower than what would be required if krill meal was added to food for its nutritional content (protein, fat, carbohydrate).

Furthermore, it is disclosed that compositions processed further into a tablet, granule, pellet, powder or treat or sprinkle capsules provide additional advantages. Inclusion of krill lipids in the form of krill meal in tablets and other compacted products provides improved stability of krill lipids and this invention discloses reduced degradation of krill lipid components including omega-3 fatty acids, astaxanthin, and phospholipids as compared to capsulated materials of the prior art.

### III. Methods of Producing Krill Meal Containing Products

The present invention is not limited to any particular method of processing krill, nor any particular type of krill meal. Any krill meal obtained by any method is contemplated.

A. Methods To Produce Krill Meal Nutritional Supplements

In one embodiment, the present invention contemplates a method for producing a nutritional supplement composition. In one embodiment, the method comprises mixing krill meal and lipid insoluble ingredient(s). In one embodiment, the method further comprises mixing suitable excipients. The composition is preferably formulated to a desired form by using methods well known for a person skilled in the art.

In one embodiment, the mixing produces a homogenous composition comprising dry krill meal and lipid insoluble ingredients. In one embodiment, the mixing of the dry krill meal includes krill oil. One advantage of the presently disclosed method is that, unlike previous methods producing unstable krill oil products (since some components, in particular phospholopids may degrade with time), the krill meal krill oil as produced herein is stable.

B. Methods to Produce Palatable Animal Food

In one embodiment, the present invention contemplates a novel method of enhancing the palatability of animal food or feed, in particular dog food by adding low levels of krill meal. Furthermore, the method further comprises increasing the food intake of an animal. Although it is not necessary to understand the mechanism of an invention, it is believed that for some animals, in particular companion animals, such as finicky dogs which commonly do not eat sufficient amounts of food offered, krill meal food supplements may improved food consumption.

In one embodiment, the method further comprises adding the krill meal composition to an animal food or feed. In one embodiment, the amount of krill meal composition added to the animal food or feed is less than 5%, preferably less than 3%, more preferably less than 1% (w/w), still more preferably less than 0.5%, most preferably less than 0.1% of the weight of the feed. In one embodiment, the amount of krill meal composition added to the animal food or feed ranges between approximately 0.01-0.9% (w/w), more preferably ranging between approximately 0.1-0.5% (w/w).

In one embodiment, a krill meal food or feed comprises a highly palatable treat used for training of the dogs or other companion animals, where krill meal is used as an animal feed ingredient.

It is a surprising feature of the presently contemplated invention that very low doses of krill meal improve food and/or feed palatability. Previously reported data on the effect of krill meal as a feed attractant has been contradictory. For example, it has been observed that krill meal can be used as a feed attractant in fish [9], while other studies report reduced feed intake in chicken fed a diet supplemented with krill meal [10].

C. Methods to Produce Krill Meal Nutritional Supplements

In one embodiment, the present invention contemplates a nutritional supplement composition comprising krill meal lipids and lipid insoluble components. In one embodiment, the nutritional supplement composition comprises a companion animal treat. In one embodiment, the nutritional supplement composition comprises a suitable form selected from the group consisting of a tablet, a capsule, a granule, a pellet, or a powder. In one embodiment, the capsule comprises a hard gelatin capsule. In one embodiment, the hard gelatin capsule is a sprinkle capsule. Although it is not necessary to understand the mechanism of an invention, it is believed that the nutritional supplement is a health promoting composition. Some advantages of the presently contemplated nutritional supplements includes, but is not limited to: i) that the combination of nutrients has synergistic effects; ii) that the nutritional supplements within the krill meal are stable; and iii) the nutritional supplement is easy to administer to human or non-human animal.

In one embodiment, the present invention contemplates a krill meal nutritional supplement comprising cartilage protecting substances. In one embodiment, the cartilage protecting substance comprises chondroitin and/or glucosamine. In one embodiment, the nutritional supplement may further comprise a lipid insoluble ingredient.

The invention is not limited to any particular lipid insoluble ingredient, but a range of lipid insoluble ingredients are contemplated. Non-limiting examples of such components are: glucosamine hydrochloride, glucosamine sulfate, glucosamine potassium, glucosamine sodium, N-acetyl d-glucosamine, chondroitin, hyaluronic acid, green lipped mussel powder, creatine, L-carnitine, ascorbic acid, manganese, manganese proteinate, zinc, zinc proteinate, copper, copper proteinate, ginseng, green tea extract, ginger, garlic, vincamine, grape seed extract, grape seed meal, dimethyl glycine, whey protein, brewer's yeast, St. John's wort, vinpocetine, aloe vera, ginko biloba, curcumin, betaglucans, mammoligosaccharides and any combination thereof.

The invention is not limited to the particular methodology, protocols, and reagents described herein because they may vary. Further, the terminology used herein is for the purpose of describing particular embodiments only and is not intended to limit the scope of the present invention.

### Experimental

### EXAMPLE 1

### Compositional Analysis of Krill Meal

Krill meal was made according to the process disclosed in [17]. Analysis showed that the meal composed of 5% water, 40% lipids , 50% peptides , 5% ash, and 403 mg/kg astaxanthin esters. The fatty acid composition and the lipid class composition of the lipid fraction were determined using standard methods in the art and the results are shown Tables 1 and 2 below.

TABLE 1
Fatty acid composition of the krill meal
made according to reference 17.

| Fatty acids | mg/g |
|---|---|
| Sum saturated | 221 |
| Sum monounsaturated | 145 |
| Sum total PUFA | 323 |
| Sum total PUFA n-3 | 294 |
| Sum total PUFA n-6 | 2.2 |
| EPA + DHA | 221 |
| EPA/DHA | 1.4 |

TABLE 2
Lipid class composition of the krill meal
made according to reference 17.

| Lipid classes | g/100 g |
|---|---|
| TG | 36 |
| DG | 0.8 |
| MG | <1 |
| FFA | 1.8 |
| Sterols | 1.9 |
| Phosphatidylcholine | 44 |
| Total neutral | 40 |
| Total polar | 53.2 |

In addition, the peptide fraction of the krill meal was subjected to size exclusion chromatography for the determination of the molecular weight distribution of the peptides (see table 3).

TABLE 3
Molecular weight distribution of peptides in the krill meal.

| Molecular weight of peptide | % of peptides |
|---|---|
| >70000 | 2.2 |
| 70000-60000 | 0.3 |
| 60000-50000 | 0.2 |
| 50000-40000 | 0.2 |
| 40000-30000 | 0.2 |
| 30000-20000 | 0.4 |
| 20000-10000 | 1.0 |
| 10000-5000 | 8.0 |
| 5000-1000 | 11.1 |
| <1000 | 76.3 |

### EXAMPLE 2

### Preparation of a Krill Meal Supplement Tablet

The krill meal made according to the method in [17] is mixed with glucosamine, chondroitin and a natural antioxidant (for example green tea extract or tocopherols) or other natural health promoting ingredients (for example curcumin) The ingredients are mixed so that the ratio of krill oil omega-3 fatty acid to glucosamine and chondroitin matches with what is considered to be beneficial to alleviate the symptoms in dogs with osteoarthritis. Here krill meal 30%, glucosamine and chondroitin 30% and excipients 30% (w/w) are mixed together. The stability and homogeneity of the product is studied.

The product is further processed into a tablet using known methods in the art. In the tablet there is at least 4% krill oil omega-3 fatty acids. A tablet and a prior art soft gelatin capsule are administered to a dog and compared, whether the dog prefers to consume the tablet over the capsule or vice versa.

### EXAMPLE 3

### Stability of a Krill Meal Nutritional Supplement Tablet

QRILL® krill meal was obtained from Aker Biomarine (Oslo, Norway) and mixed with glucosamine, chondroitin, a natural antioxidant (vitamin-C) and curcumin.

The ingredients were mixed so that the ratio of krill oil omega-3 fatty acid to glucosamine and chondroitin matched with what is considered to be beneficial to alleviate the symptoms in dogs with osteoarthritis.

Here, krill meal 30%, glucosamine and chondroitin 30% and excipients 30% (w/w) were mixed together and processed into a tablet using known methods in the art. The product was found to be homogenous with respect to the oil content, but it was not stable with respect to the oil quality. The level of free fatty acids and lyso-phospholipids increased gradually over time.

A tablet and a prior art soft gelatin capsule filled with krill oil were administered individually to a group of ten dogs. It was found that 9 out of 10 dogs ate the tablets, none of the dogs ate the capsule voluntarily.

### EXAMPLE 4

### Comparative Stability of Krill Meal Tablets Versus Krill Oil

Tablets are made according to Example 1 and Example 2. A krill oil product is obtained (Neptune Biotech, Laval, Canada) and combined with commercially available glucosamine and chondroitin into a liquid product. The chemical stability of the three products is studied and compared.

After a certain amount of time, the levels of lyso-phospholipids, omega-3 fatty acids, peroxide value, p-anisidine value andastaxanthin in the tablets are compared with their levels in the liquid krill oil product. Krill oil and glucosamine +chondroitin are mixed in ratios 4:1, 1:1 and 1:4 and it is studied whether the lipid (krill oil) and non-lipid (glucosamine+chondroitin) phase are separated. In addition, krill meal and glucosamine+chondroitin are mixed in ratios 4:1, 1:1 and 1:4 based on the oil content of krill meal and a tablet using methods known in the art is produced.

The homogeneity and stability of the krill meal and glucosamine+chondroitin tablets is compared to the homogeneity and stability of the capsulated krill oil glucosamine+chondroitin mixture. It is to be observed that the liquid krill oil products result on a phase separation and is therefore not homogenous. It is also to be observed that the liquid krill oil product compared to the tablets results in higher levels of lyso-phospholipids, lower omega-3 levels, higher peroxide values, higher p-anisidine values and lower Astaxanthin values. These results clearly show that there is an improved chemical and physical stability of the krill tablets compared to the krill oil product.

### EXAMPLE 5

### Palatability of Krill Meal Nutritional Supplements

Tablets made in accordance with Example 2 and Example 3 were tested for palatability with 10 dogs. Control product was a same size tablet containing only glucosamine and chondroitin as active ingredients.

Palatability test was performed as follows: each dog received three products [with krill meal (Krill 1 and Krill 2) and one without krill meal (Control)]. Tablets were administered daily with food in the morning at 1 tablet/10 kg body weight for 30 days. Order of the tablets was random, but it was recorded with observations on palatability.

No difference was found in palatability between Krill 1 and Krill 2, but the Control was clearly less palatable. Often the Control tablet remained in the bowl after the food was consumed and had to be fed directly into the mouth of the dog.

### EXAMPLE 6

### Palatability of Commercially Available Krill Meal Food Additives

QRILL® Krill meal was obtained from Aker Biomarine (Oslo, Norway) and added to dry animal food at four different levels of inclusion: 5%, 1%, or 0.1% (w/w) level and one formulation without any krill meal (0% or Control).

These four foods were offered to a group of dogs to determine the relative palatability of foods with krill meal versus Control. The palatability test was performed as a ‘two-bowl-test’ where each of the dogs were offered one bowl of food with krill meal (5, 1 or 0.1%) and one bowl of food without krill meal (Control). The amount of food in each of the bowls was roughly 50% of the daily allowance of the dog. Dogs were allowed to eat until one of the bowls was empty or until both of the bowls were half empty. The preference was recorded and ‘no preference’ was recorded in case the dogs ate from both bowls.

In most cases the foods with krill meal were more palatable. There was no clear effect of the inclusion level. Roughly 20% of dogs did not show preference, but ate from both bowls. Very few dogs preferred the Control food suggesting that adding krill meal to the food enhanced the palatability of the food.

### EXAMPLE 7

### Palatability of Fresh Krill Meal Food Additives

Krill meal was made according to reference [17] and added to dry animal food at four different levels of inclusion: 5%, 1%, or 0.1% (w/w) level and one formulation without any krill meal (0% or Control).

These four foods were offered to a group of dogs to determine the relative palatability of foods with krill meal versus Control. The palatability test was performed as a ‘two-bowl-test’ where each of the dogs were offered one bowl of food with krill meal (5, 1 or 0.1%) and one bowl of food without krill meal (Control). The amount of food in each of the bowls was roughly 50% of the daily allowance of the dog. Dogs were allowed to eat until one of the bowls was empty or until both of the bowls were half empty. The preference was recorded and ‘no preference’ was recorded in case the dogs ate from both bowls.

In most cases the foods with krill meal were more palatable. There was no clear effect of the inclusion level. Roughly 20% of dogs did not show preference, but ate from both bowls. Very few dogs preferred the Control food suggesting that adding krill meal to the food enhanced the palatability of the food.

### EXAMPLE 8

### Preparation of Healthy Companion Animal Treats

A healthy treat comprising krill lipids in the form of krill meal and cartilage protecting substances is prepared. The formula of healthy treat combines 2000 mg of krill meal, 500 mg of glucosamine+chondroitin and 2500 mg of base ingredients for a total weight of 5 g/treat. Base ingredients consist of meat meals and extracts, other animal by-products, fish meal and extracts, plant protein meals, cereal meals or fractions of cereals and various binding agents known in the art.

Pet treats may also contain additional ingredients that provide desirable consistency and bulk to the product. Alternatively, the 5 g treat can be produced from krill meal and cartilage protecting substances alone by mixing 4500 mg of krill meal+binding agents with 500 mg of glucosamine+chondroitin. After mixing the ingredients the mixture is pelleted, extruded or otherwise processed into treats of different shapes.

### EXAMPLE 9

### Preparation of Krill Meal Hard Gelatin Capsules

The mixture of ingredients described in accordance with Example 1 are used as a filling of hard gelatin capsules (e.g., sprinkle capsules). The capsule is opened and its contents sprinkled over a bowl of food. The palatability of the capsules is assessed and compared with control capsules containing no krill meal.

### EXAMPLE 10

### Krill Meal Nutritional Supplement Treatment of Degenerative Joint Disease

A nutritional supplement for dogs comprising krill meal and cartilage protecting substances was prepared in accordance with Example 8 with the exception of the relative ingredient percentages. The supplement combined 90% of krill meal (made according to ref 17) and 10% glucosamine hydrochloride +chondroitin sulfate (in 60:40 ratio). After mixing the ingredients were pelleted with a matrix type granulation unit and stored in aluminum foil plastic bags under nitrogen atmosphere. This supplement was used in a clinical test involving 10 dogs with degenerative joint disease (i.e., for example, osteoarthritis; OA). The test was performed by a veterinary clinic.

Ten dogs of various ages and breeds presenting with different types of joint problems resulting in various degrees of OA were recruited to participate in a 6 week test. OA is often associated with pain and thus it influences the movement of the dog. Before starting the administration of the supplement the dogs were brought to the veterinary clinic for examination which included weighing, body condition scoring, palpation of the joints and observations on the movement of the dogs. A force plate (4LegCheck; ReDog, Väster\dot{a}s, Sweden) was used to determine the distribution of weight between the four legs while the dog was standing. Deviation from equal distribution of weight (=5%) between the left and right leg suggests pain/lameness. Also deviation from the typically observed 60:40 distribution of weight (±5%) between front and rear legs is considered to be a sign of lameness. The dogs were kept at home and supplemented with the food supplement for six weeks. The dose of the supplement was 5 g/10 kg of body weight.

After six weeks the dogs returned to the clinic and the clinical examination was repeated. In addition to the clinical re-examination the owners filled in a questionnaire regarding the mobility of the dog by using a modified Helsinki Chronic Pain Index (HCPI) questionnaire (Hielm-Björkman et al. 2003) with 11 points involving movement of the dog by using scores ranging from 1 to 5 where 1 represents greatly reduced mobility, 3 represents no change and 5 represents greatly improved mobility. Mean of the 11 points scored from 1 to 5 were used as the mobility index. Thus, three types of observations regarding the change in dogs mobility were performed: one based on observations made by the clinician, one based on the assessment of the owner performed by using a validated questionnaire and one based on using a device providing highly repeatable measurements on lameness based on weight distribution between the four legs of the dog.

All three measurements described above provided evidence on consistent improvement in the mobility of the dogs during the six week nutritional supplementation period. Briefly, force plate measurements indicated that 7 out of 10 dogs demonstrated reduced lameness due to supplementation. Also, owner assessed average mobility score improved in 8 out of 10 cases with mobility indexes ranging from 3.5 to 5. In one dog there was no change and in one dog the changes were both positive and negative resulting in overall score of 3. This dog demonstrated significant improvement in mood, activity and playing. However, increased mobility was associated with greatly increased difficulty in moving after rest. Finally, the clinical examination by the veterinarian documented 6 cases of improved movement, 5 cases of reduced pain and one case of reduced lameness and pain, respectively. In total, 8 out of 10 dogs demonstrated improvement in one or more clinical endpoints.

It is of particular interest to examine the supplementation of the dogs prior to the test. One dog had been supplemented with a combination of omega-3 fish oil and a joint health product (Seraquin by Boehringer Ingelheim), four dogs had been supplemented with a joint health product (Cartivet by Biofarm and Arthroflex by ScanVet) and five dogs had received no supplementation. Seraquin, Cartivet and Arthroflex are products that contain glucosamine and chondroitin as the main chondroprotective nutrients. All five dogs receiving nutritional supplements prior to the test start responded with significant improvement in joint health and mobility suggesting that the combination of krill meal and chondroprotective nutrients (glucosamine+chondroitin) as in the current composition and applied during the 6 week test period was more effective than fish oil+chondroprotective nutrients (Seraquin) or chondroprotective ingredients alone (Cartivet, Arthroflex).

### EXAMPLE 11

### Krill Meal Treatment of Degenerative Joint Disease

A nutritional supplement for dogs comprising krill meal alone was prepared having the ingredients described in accordance with Example 1. Krill meal prepared according to ref. 17 was pelleted with a matrix type granulation unit and stored in aluminum foil plastic bags under nitrogen atmosphere. Pelleted krill meal was used as a supplement in a clinical test involving 8 dogs with degenerative joint disease (i.e., for example, osteoarthritis; OA). The test was performed by a veterinary clinic.

Eight dogs of various ages and breeds presenting with different types of joint problems resulting in various degrees of OA were recruited to participate in a 6 week test.

Before starting the administration of the supplement the dogs were brought to the veterinary clinic for examination which included a clinical examination, measurements on a force plate and owner assessment of the mobility as described in Example 10. Supplement was fed to the dogs at 5 g/10 kg of body weight.

All three measurements of pain and mobility provided evidence on improvement. However, the overall improvement was somewhat less than what was observed for the combination of krill meal and glucosamine+chondroitin (Example 9). Briefly, force plate measurements demonstrated significant reduction in lameness in 2 out of 8 dogs. Qwner assessed average mobility score improved in 5 out of 8 cases with mobility indexes ranging from 3.25 to 4.5. In 3 out of 8 cases there was no change. Finally, the clinical examination by the veterinarian documented 2 cases of improved movement, 1 case of reduced pain, 1 case of reduced lameness and 2 cases of reduced stiffness. In total, 4 out of 8 dogs demonstrated improvement in one or more clinical endpoints. In this group the dogs demonstrated, in general, less symptoms at the baseline and their forceplate measurements also indicated less lameness. Thus, the apparent lower efficacy of the krill meal supplement compared to the combination supplement (Example 9) may be influenced by the random selection of less symptomatic test subjects into this group.

It is also of particular interest to examine the supplementation of the dogs prior to the test with krill meal alone. One dog had been supplemented with a combination of omega-3 fish oil and a joint health product (Seraquin by Boehringer Ingelheim), four dogs had been supplemented with a dry dog food containing chondroprotective ingredients (fish oil omega-3 fatty acids, greenlipped mussel meal and glucosamine +chondroitin; Royal Canine Mobility formula) and three dogs had received no supplementation. Three dogs receiving nutritional supplements prior to the test start responded with significant improvement in joint health and mobility and two dogs showed no response suggesting that krill meal alone is in some cases equally or more effective than supplements containing fish oil and/or chondroprotective nutrients (glucosamine+chondroitin).

### References

[1] U.S. Pat. No. 6,054,147
[2] U.S. Pat. No. 5,843,919
[3] WO 2007/144381
[4] US 2005/0101563
[5] Deutsch L. (2007). Evaluation of the effect of Neptune Krill Oil on chronic inflammation and arthritic symptoms. J. Am. Coll. Nutr. 2007 February; 26(1):39-48.
[6] US 20070113796.
[7] U.S. Pat. No. 6,020,003
[8] U.S. Pat. No. 6,800,299
[9] US 20080274203.
[10] R. E. Olsen, J. Suontama, E. Langmyhr, H. Mundheim, E. Ringo, W. Melle, M. K. Malde, G.-I. Hemre, Aquaculture Nutrition 12 (4) 280-290, 2006.
[11] R. Rys and J. Koreleski. Arch Tiemahr 29 (3) 181-188, 1979.
[12] US 20090061067
[13] US 20030113432
[14] Krill Canada, Langley, Canada and Dynamic Aqua-Supply Ltd, Surrey, Canada
[15] Japanese Patent Publication No. 57-11876
[16] US 20060035313
[17] PCT Application WO2010030193. Process for reducing the fluoride content when producing proteinaceous concentrate from krill.

1. A nutritional supplement composition comprising krill meal and lipid insoluble ingredients.

2. The composition according to claim 1, wherein said lipid insoluble ingredient comprises cartilage protecting substances.

3. The composition of claim 2, wherein said cartilage protecting substances are glucosamine and chondroitin.

4. The composition of claim 1, wherein said lipid insoluble ingredients are selected from the group consisting of glucosamine hydrochloride, glucosamine sulfate, glucosamine potassium, glucosamine sodium, N-acetyl d-glucosamine, chondroitin, chondroitin sulfate, curcumin, ascorbic acid, hyaluronic acid, green lipped mussel powder, creatine, L-carnitine, ascorbic acid, manganese, manganese proteinate, zinc, zinc proteinate, copper, copper proteinate, ginseng, green tea extract, ginger, garlic, vincamine, grape seed extract, grape seed meal, dimethyl glycine, whey protein, brewer's yeast, St. John's wort, vinpocetine, aloe vera, ginko biloba, curcumin, betaglucans, and mannaoligosaccharides.

5. The composition of claim 1, wherein said composition comprises a krill meal to lipid insoluble ingredients weight ratio ranging between approximately 16:1 to 1:1.

6. The composition of claim 1, wherein said composition further comprises a lipid soluble ingredient.

7. omposition of claim 4, wherein said lipid soluble ingredient is selected from the group consisting of vitamin A, vitamin D, vitamin E, alpha lipoic acid, lutein, and natural carotenoids.

8. The composition of claim 1, wherein the composition comprises 10-90% (w/w) krill meal, 10-90% (w/w) lipid insoluble ingredient(s), and 0-80% (w/w) excipients.

9. A composition comprising an animal food and a krill meal food ingredient.

10. The composition of claim 9, wherein the krill meal food ingredient is less than 5% of said composition.

11. The composition of claim 9, wherein the krill meal food ingredient is less than 1% of said composition.

12. A method, comprising:
a) providing:
i) an animal exhibiting at least one symptom of a degenerative joint disease;
ii) a nutritional supplement composition comprising krill meal and a lipid insoluble ingredient;
b) administering said composition to said animal under conditions such that said at least one symptom is reduced.

13. The method of claim 12, wherein said administered composition comprises krill meal ranging between 0.005-0.50 grams per day per kilogram of said animal's body weight.

14. The method of claim 13, wherein said degenerative joint disease comprises osteoarthritis.

15. The method of claim 12, wherein said lipid insoluble ingredient comprises cartilage protecting substances.

16. The method of claim 15, wherein said cartilage protecting substances are glucosamine and chondroitin.

17. A method, comprising:
a) providing:
i) an animal in need of increased food intake;
ii) an animal food or feed comprising between approximately 0.01% (w/w) and 1.0% (w/w) krill meal;
b) feeding said animal said food under conditions such that said animal's food intake increases.

18. The method of claim 17, wherein said method further comprises improving the health of said animal.

19. The method of claim 17, wherein said animal comprises a non-human animal.

20. The method of claim 19, where said non-human animal comprises a dog.
